(19) United States
(12) Patent Application Publication
(10) Pub. No.: US 2025/0020673 A1
(43) Pub. Date: Jan. 16, 2025

(54) COMPOSITIONS AND METHODS FOR ASSAYING GLUTATHIONE RECYLCLING CAPACITY

(71) Applicant: Lankenau Institute for Medical Research, Wynnewood, PA (US)

(72) Inventors: U. Margaretha Wallon, Ardmore, PA (US); Nolan L. Metz, Philadelphia, PA (US)

(21) Appl. No.: 18/706,626

(22) PCT Filed: Nov. 3, 2022
(86) PCT No.: PCT/US2022/079255

§ 371 Date: May 1, 2024

Related U.S. Application Data
(60) Provisional application No. 63/275,279, filed on Nov. 3, 2021.
(60) Provisional application No. 63/275,271, filed on Nov. 3, 2021.

(51) Int. Cl. G01N 33/80; G01N 1/40

(52) U.S. Cl. CPC G01N 33/80; G01N 1/4077; G01N 2800/52; G01N 2800/7009

(57) ABSTRACT

A methods and compositions for assessing the recycling capacity of glutathione in biological samples.

### BACKGROUND OF THE INVENTION

Chemotherapy-induced peripheral neuropathy (CIPN) is a disabling side-effect of platinum-based chemotherapies, like cisplatin, oxaliplatin and carboplatin. While not all patients experience this side-effect, those who do are at risk for lifelong chronic neuropathy.

Cancer survivorship issues are growing in parallel with positive outcomes in cancer treatment. As a result, attention to the diagnosis, prognosis and treatment of CIPN cases are increasing. The prevalence of CIPN resulting from different antitumor drugs and their doses varies significantly. Indeed, while approximately 70% of patients receiving chemotherapy develop CIPN during the first month of treatment, approximately 20-30% of these patients experience conversion to chronic CIPN at 6 months or later after cessation of chemotherapy. Importantly, the symptoms of these delayed complications may not only persist for several months but also be progressively aggravated. Cases in which mild neuropathy worsens or a new form of CIPN develops has been termed ‘coasting’. Since chemotherapeutics are not being administered when coasting develops, this disorder poses a great challenge to clinicians, since patients may be cancer-free but still suffer from neuropathy caused by their earlier cancer treatment.

As pharmacological methods are developed for prevention or treatment, compositions and methods to identify patients at highest risk of chronic CIPN are also crucial to develop.

### SUMMARY OF THE INVENTION

Described herein are assays and methods of uses thereof that facilitate large-scale processing of patient samples to measure GSH recycling capacity.

In one aspect, provided herein is an assay for measuring GSH recycling capacity in a collection of samples containing RBC that includes (a) combining each of the samples with a solution containing HEDS to obtain a collection of first volumes, and incubating said collection of first volumes; (b) centrifuging the samples to remove RBCs and debris from suspension; (c) obtaining an aliquot of each of the said first volumes, substantially free of RBC and debris, and admixing each aliquot with trichloroacetic acid (TCA) to obtain a collection of second volumes; (d) subjecting the collection of second volumes, or aliquots thereof, to centrifugation; (e) obtaining a collection of supernatants from the second volumes, and combining each of the supernatants, or aliquots thereof, with a solution containing 5,5′-Disulfanediylbis(2-nitrobenzoic acid) (DNTB) to obtain a collection of third volumes; and (f) subjecting the collection of third volumes, or aliquots thereof, to spectrophotometric analysis to obtain a series of absorbance readings, wherein the absorbance readings obtained in (e) are indicative of GSH recycling capacity in the sample containing RBC.

In certain embodiments, step (a) includes obtaining a collection of first volumes in a series of 8-well strips of wells, optionally wherein each strip contains a different sample or dilutions of different samples. In certain embodiments, the series of 8-well strips include one or more wells containing a positive control containing L-Cysteine hydrochloride monohydrate (LCHM), optionally in lyophilized form or in a solution at a concentration of about 25 μMol, about 50 μMol, and/or about 25 μMol to about 50 μMol. In certain embodiments, the 8-well strips include two or more different LHCM positive, each being a different amount or concentration of LCHM.

In one aspect, provided herein is an assay for measuring glutathione (GSH) recycling capacity in a sample containing red blood cells (RBC) that includes, (a) combining the sample with a solution containing hydroxy-ethyl-disulfide (HEDS) to obtain a first volume, and incubating said first volume to allow a substantial fraction of RBC from the sample to sediment;
(b) obtaining an aliquot of the first volume, substantially free of RBC, and admixing said aliquot of the first volume with a solution containing magnetic nanobeads to obtain a second volume; (c) subjecting the second volume, or an aliquot thereof, to a magnetic field; (d) obtaining a supernatant from the second volume, and combining the supernatant, or aliquot thereof, with a solution containing 5,5′-Disulfanediylbis(2-nitrobenzoic acid) (DNTB) to obtain a third volume; and (e) subjecting the third volume, or aliquot thereof, to spectrophotometric analysis to obtain an absorbance reading, wherein the absorbance measured in (e) is indicative of GSH recycling capacity in the sample containing RBC.

In one aspect, provided herein is an assay for measuring GSH recycling capacity in a collection of samples containing RBC that includes (a) combining each of the samples with a solution containing HEDS to obtain a collection of first volumes, and incubating said collection of first volumes to allow a substantial fraction of RBCs from the sample to sediment; (b) obtaining an aliquot of each of the said first volumes, substantially free of RBCs, and admixing each aliquot with a solution containing magnetic nanobeads to obtain a collection of second volumes; (c) subjecting the collection of second volumes, or aliquots thereof, to a magnetic field;
(d) obtaining a collection of supernatants from the second volumes, and combining each of the supernatants, or aliquots thereof, with a solution containing 5,5′-Disulfanediylbis (2-nitrobenzoic acid) (DNTB) to obtain a collection of third volumes; and (e) subjecting the collection of third volumes, or aliquot thereof, to spectrophotometric analysis to obtain a series of absorbance readings, wherein the absorbances measured in (e) are indicative of GSH recycling capacity in the samples containing RBC.

In a further aspect, kits for performing assays for measuring glutathione (GSH) recycling capacity in a biological sample containing red blood cells are provided.

Other aspects and advantages of these compositions and methods are described further in the following detailed description of the preferred embodiments thereof.

### BRIEF DESCRIPTION OF THE DRAWINGS

FIG. 1 provides a table with grading criteria for nervous system disorders.

FIG. 2 shows results from an analysis comparing age of patients with an average CIPN grade.

FIG. 3 shows results from an analysis of Chemotox scores comparing gender of patients and time of year.

FIG. 4 shows results from an analysis utilizing a minimum of four different collection timepoints to determine Chemotox scores versus average CIPN grade for patients.

FIG. 5 shows the ability to predict grade 3 CIPN versus grade 0 CIPN using patient baseline/pre-treatment Chemotox scores.

FIG. 6 shows the ability to predict grade 3 CIPN versus grade 0 CIPN using patient Chemotox scores determined following a first treatment but prior to a second treatment (“pre 2”).

FIG. 7 shows the ability to predict grade 3 CIPN versus grade 0 CIPN using patient Chemotox scores obtained from a collection of four time points.

FIG. 8 shows a graph with ROC curves demonstrating that an approach utilizing multiple Chemotox scores during treatment outperforms the methods using only a baseline/pre-treatment assessment to predicted CIPN. The total number of patients used for analyses is 260 (N=260). Using the mean of the first four test samplings, the ROC is approaching 0.8.

FIG. 9A and FIG. 9B show average Chemotox scores in patients versus CIPN severity using Chemotox scores obtain at four different time points (FIG. 8A) or two different time points (FIG. 9B).

FIG. 10 provides a graph showing examples of variation in individual patients and relative changes in Chemotox scores during treatment.

FIG. 11A and FIG. 11B show the accuracy in predicting grade 3 CIPN vs grade 0 in patients receiving a taxol containing treatment (FIG. 11A) or a combination oxaliplatin/FOLFOX treatment (FIG. 111B) using Chemotox scores obtained from four timepoints.

FIG. 12 shows an eight well strip as utilized in the high-throughput assays provided herein. The eight well strips can be loaded (up to twelve) into a frame that holds the samples during incubation, centrifugation, and absorbance readings steps.

FIG. 13 shows results from parallel analysis of samples using the high-throughput plate-based assay provided herein and a previously described tube-based method.

### DETAILED DESCRIPTION

Methods and compositions described herein to measuring recycling of the antioxidant glutathione (GSH) in blood.

The present inventors have shown that measurements of recycling of the antioxidant glutathione (GSH) in blood is predictive of CIPN or severity of CIPN in a subject. In particular, it has been demonstrated that longitudinal changes in a patient's blood before and after the administration of chemotherapy can be used to identify a patient with an elevated risk of CIPN. Specifically, if levels of the baseline GSH recycling capacity remain lower and do not rebound during multiple cycles of chemotherapy, the patient is at increased risk of CIPN.

While not wishing to be bound by theory, platinum-based therapies cause bursts of reactive oxygen species (ROS) which can trigger structural changes in peripheral nerves including neuronopathy, axonopathy and/or myelinopathy. Glutathione, a natural antioxidant in the body, plays an important role in reduction-oxidation (redox) homeostasis. It has been hypothesized that changes in GSH recycling capacity that occur in some patients following chemotherapy are predictive for CIPN for the following reasons. Reactive oxygen species (ROS) are known to destroy various tissues and cellular components. Neuropathy is caused by damage to ion channels, microtubules, dorsal root ganglion, small nerve fibers, mitochondria, and myelin, and ROS are known to damage mitochondria, myelin sheets, and calcium ion homeostasis. GSH is a key antioxidant for neutralizing ROS. When ROS are neutralized by GSH, the latter becomes oxidized to a dimeric form (GSSG) that needs to be recycled to its reduced monomeric form (GSH) that can neutralize more ROS. Thus, if more ROS are produced than there is GSH available to neutralize them, tissue damage may occur. Why some patients are able to efficiently recycle GSSG and sustain GSH levels during chemotherapy at levels sufficient to quench ROS, while others cannot, is unclear.

The inventors determined that a patient's risk of CIPN may reflect naturally occurring individual variations in the ability of GSH to be recycled efficiently to scavenge free radicals during the period chemotherapy is administered to cancer patients. Redox homeostasis as maintained by GSH recycling is used as a biomarker assayed during chemotherapy to predict risks of chronic CIPN in an individual.

GSH activity is known to be a critical determinant in the capability of cells to survive bursts of free radicals generated by chemotherapy treatment. Therefore, effective GSH recycling is crucial for cells to detoxify ROS and minimize tissue damage. The inventors identified a relationship between the redox homeostasis as maintained by glutathione (GSH) with susceptibility to chronic CIPN in subjects.

Currently, there is no approved pharmacological means to treat or prevent CIPN. Only dose reduction, delaying, or discontinuing potentially life-saving chemotherapy can limit the development and progression of CIPN. However, many cancer patients and oncologists pay increasing attention to risks of CIPN, as research to mitigate or prevent this side-effect continues. Accordingly, there is great interest in identifying patients most at risk, and where appropriate, altering or adding treatment regimens to eliminate CIPN or reduce its severity.

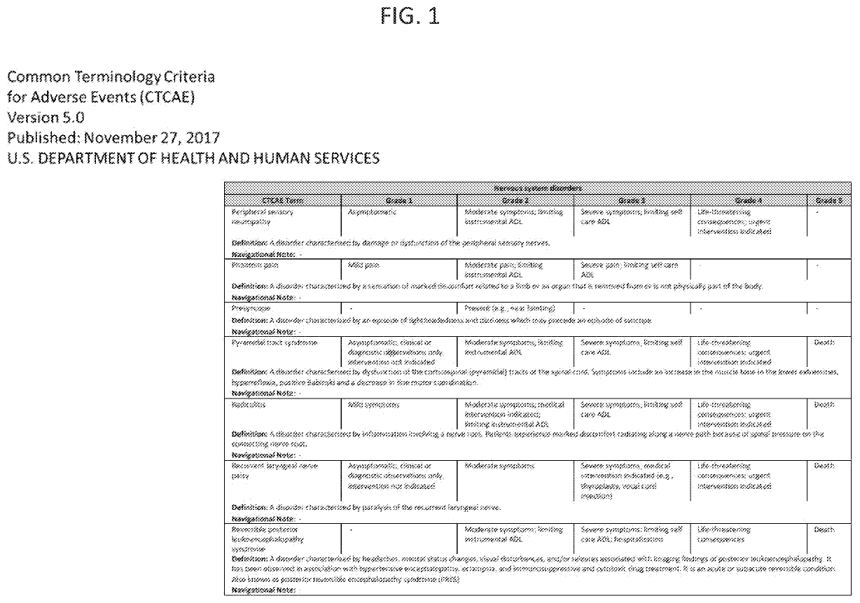

As used herein the term “chemotherapy-induced peripheral neuropathy” or “CIPN” refers to or describes symptoms arising from damage to peripheral nerves in a patient being treated or having been treated with chemotherapy. Depending on the nerves affected, symptoms include, without limitation, tingling (“pins and needles”), pain, burning sensations, decreased sensation, increased sensitivity to touch, temperature, pressure, or pain, loss of feeling (can be numbness or a reduced ability to sense pressure, touch, heat, or cold), trouble using fingers to pick up or hold things, dropping things, balance problems, trouble with tripping or stumbling while walking, pressure or temperature hurt more than usual (e.g., cold sensitivity), shrinking muscles, muscle weakness, trouble swallowing, constipation, trouble passing urine, blood pressure changes, and altered nerve conduction velocity with decreased or no reflexes. A grading scale can be utilized to assess the severity of CIPN (see, e.g., FIG. 1). See also, Zhang et al. Biomed Rep. 2017 March; 6(3): 267-271, which is incorporated herein by reference).

As used herein, “hyperalgesia” refers to an increased sensitivity to pain, which may be caused by damage to nociceptors or peripheral nerves (i.e., neuropathy). The term refers to temporary and permanent hyperalgesia, and encompasses both primary hyperalgesia (i.e., pain sensitivity occurring directly in damaged tissues) and secondary hyperalgesia (i.e., pain sensitivity occurring in undamaged tissues surrounding damaged tissues). The term encompasses hyperalgesia caused by, but not limited to, neuropathy caused by, resulting from, or otherwise associated with drug toxicity. In some embodiments hyperalgesia is caused by chemotherapy-induced peripheral neuropathy.

In one aspect, a method for assessing susceptibility to CIPN comprises assaying a mammalian subject's biological sample containing red blood cells (RBC) for a level of oxidative stress. Oxidative stress is essentially an imbalance between the production of free radicals and the ability of the body to counteract or detoxify their harmful effects through neutralization by antioxidants. In one embodiment, an assay is performed by assessing the GSH recycling activity of the RBC in the sample as an indicator of the quality in the antioxidant response to oxidative stress. This assay is based on the discovery that a reduction of GSH recycling capacity in a subject's RBC (i.e., a relative reduction in the ability to survive high oxidative stress) during chemotherapy was inversely associated with higher risk of the subject developing CIPN.

According to the methods described herein, the biological sample is obtained from a mammalian subject prior to and following administration of a chemotherapeutic agent. By “biological sample” or “sample” as referred to herein, is meant a biological fluid containing red blood cells. In certain embodiments, the biological sample is whole blood. In certain embodiments, the sample is another fluid containing RBCs. In certain embodiments, the sample is diluted. In certain embodiments, the sample is a concentrated sample.

By “patient” or “subject” as used herein means a mammalian animal, including a human, a veterinary or farm animal, a domestic animal or pet, and animals normally used for clinical research such as mice and rats. More specifically, the subject of these methods and compositions is a human. In certain embodiments, the subject has a cancer.

A “carrier” as used herein refers to, for example, a diluent, matrix, adjuvant, preservative (e.g., Thimersol, benzyl alcohol), anti-oxidant (e.g., ascorbic acid, Sodium metabisulfite), solubilizer (e.g., Tween 80, Polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention can be maintained. Carriers can be sterile liquids, such as water and oils, including those of petroleum, animal, vegetable or synthetic origin. Water or aqueous saline solutions and aqueous dextrose and glycerol solutions may be also employed as carriers.

By “chemotherapeutic agent” as used herein refers to any compound (including its derivatives) which may be used to treat cancer. Chemotherapeutic agents (e.g., anti-cancer agents) are well known in the art and include, but are not limited to, anthracenediones (anthraquinones) such as anthracyclines (e.g., daunorubicin (daunomycin; rubidomycin), doxorubicin, epirubicin, idarubicin, and valrubicin), mitoxantrone, and pixantrone; platinum-based agents (e.g., cisplatin, carboplatin, oxaliplatin, satraplatin, picoplatin, nedaplatin, triplatin, and lipoplatin); tamoxifen and metabolites thereof such as 4-hydroxytamoxifen (afimoxifene) and N-desmethyl-4-hydroxytamoxifen (endoxifen); taxanes such as paclitaxel (taxol) and docetaxel; alkylating agents (e.g., nitrogen mustards such as mechlorethamine (HN2), cyclophosphamide, ifosfamide, melphalan (L-sarcolysin), and chlorambucil); ethylenimines and methylmelamines (e.g., hexamethylmelamine, thiotepa, alkyl sulphonates such as busulfan, nitrosoureas such as carmustine (BCNU), lomustine (CCNLJ), semustine (methyl-CCN-U), and streptozoein (streptozotocin), and triazenes such as decarbazine (DTIC; dimethyltriazenoimidazolecarboxamide)); antimetabolites (e.g., folic acid analogues such as methotrexate (amethopterin), pyrimidine analogues such as fluorouracil (5-fluorouracil; 5-FU), floxuridine (fluorodeoxyuridine; FUdR), and cytarabine (cytosine arabinoside), and purine analogues and related inhibitors such as mercaptopurine (6-mercaptopurine; 6-MP), thioguanine (6-thioguanine; 6-TG), and pentostatin (2′-deoxycofonnycin)); natural products (e.g., vinca alkaloids such as vinblastine (VLB) and vincristine, epipodophyllotoxins such as etoposide and teniposide, and antibiotics such as dactinomycin (actinomycin D), bleomycin, plicamycin (mithramycin), and mitomycin (mitomycin Q); enzymes such as L-asparaginase; biological response modifiers such as interferon alpha); substituted ureas such as hydroxyurea; methyl hydrazine derivatives such as procarbazine (N-methylhydrazine; MIH); adrenocortical suppressants such as mitotane (o,p′-DDD) and aminoglutethimide; analogs thereof derivatives thereof and combinations thereof. Still other cytostatic chemotherapeutics known to the art may be useful in the methods described herein. See, for example, the chemotherapies described in U.S. Pat. No. 9,186,357, incorporated by reference.

In certain embodiments, the method includes administering to the subject a chemotherapeutic agent that is a taxane, a platinum compound, optionally oxaliplatin or cisplatin, a vinca alkaloid, thalidomide, epothilone, eribulin, ipilimumab, pembrolizumab, nivolumab, or bortezomib.

As used herein the term “cancer” refers to or describes the physiological condition in mammals that is typically characterized by unregulated cell growth. In one embodiment, the term “cancer” means any cancer characterized by the presence of a solid tumor. In another embodiment, a cancer is a hematological cancer. When referred to herein, a cancer includes, without limitation, melanoma, breast cancer, brain cancer, colon/rectal cancer, lung cancer, ovarian cancer, adrenal cancer, anal cancer, bile duct cancer, bladder cancer, bone cancer, endometrial cancer, esophagus cancer, eye cancer, kidney cancer, laryngeal cancer, liver cancer, head and neck cancer, nasopharyngeal cancer, osteosarcoma, oral cancer, ovarian cancer, pancreatic cancer, prostate cancer, rhabdomosarcoma, salivary gland cancer, stomach cancer, testicular cancer, thyroid cancer, vaginal cancer, lung cancer, lymphoma, myeloma, and neuroendocrine cancer.

By “chemotherapy regimen” as used in the methods described herein is generally meant either the combined or sequential administration of 1 or 2 different chemotherapeutic agents or the combined or sequential administration of 3 to 10 different chemotherapeutic agents. In the methods described herein, selection of the ≤2 drug regimen (which can include no chemotherapeutic agents) or selection of the ≥3 drug regimen depends upon the oxidative level of the blood sample. Where a sample from the subject exhibits a high glutathione (GSH) recycling dependent antioxidant activity (i.e., high oxidative stress), a chemotherapeutic regimen can be altered to include greater numbers of chemotherapeutic agents in the regimen. Where a sample from the subject exhibits a low glutathione (GSH) recycling dependent antioxidant activity (i.e., low oxidative stress), a drug regimen can be altered to include lesser numbers of chemotherapeutic agents in the regimen or even eliminate the chemotherapeutic drug regimen earlier in treatment or dispense with it as unnecessary. In certain embodiments, chemotherapy regimen includes a combination of folinic acid (leucovorin, FOL), fluorouracil (5-FU, F), and oxaliplatin (Eloxatin, OX) (i.e., FOLFOX). In certain embodiments, chemotherapy regimen includes a combination of R-CHOP (rituximab, cyclophosphamide, doxorubicin hydrochloride/hydroxydaunomycin, vincristine sulfate/Oncovin, and prednisone). In certain embodiments, chemotherapy regimen includes ABVD (doxorubicin hydrochloride/Adriamycin, bleomycin, vinblastine sulfate, and dacarbazine). In certain embodiments, chemotherapy regimen includes R-CVP (rituximab, cyclophosphamide, vincristine sulfate/Oncovin, and prednisone).

Compounds that are “administered together” or “in combination” may be administered as part of the same composition, or may be administered separately, at the same or at separate times, in the same therapeutic regimen.

By “glutathione recycling activity” is meant how well RBC can convert the tripeptide GSH from its oxidized state to a reduced state and thereby neutralize ROS. A relatively higher GSH recycling activity protects cells from oxidative stress and ROS by recycling the oxidized isoform glutathione disulfide (GSSG) to the reduced isoform glutathione monomer (GSH). Suitable GSH recycling-dependent antioxidant activity is measurable in the biological samples of these methods, in one embodiment, by the use of the OxPhos™ Cell Survival Kit, cat. no. KLD-02, Rockland Inc. This assay uses hydroxyethyl disulfide (HEDS) as an indirect indictor of glutathione-dependent detoxification involving conversion of GSH→GSSG→GSH which releases ß-mercaptoethanol (ME). In certain embodiments, the method employs the measurement of GSH recycling activity by quantifying the amount of ß-mercaptoethanol (ME) released by the blood sample treated with the reagent and spectophotochemically measuring absorbance readings of ME; converting absorbance readings into ME concentrations; normalizing ME concentrations to total red blood cell count at the time of blood draw; and measuring the GSH recycling dependent antioxidant activity of intact erythrocyte cells in the sample. In the Ox-Phos™ assay, the pre-mixed dithiobisnitrobenzoic acid (DNTB) reagents are used to spectrophotometrically determine the conversion of HEDS into ME and absorbance readings are converted into ME concentrations and normalized to total red blood cell count (RBC×10⁶), as determined at the time of blood draw. In certain embodiments, GSH dependent antioxidant activity is calculated using the conversion factor provided with the OxPhos™ assay protocol and normalized to the total red blood cell count. In certain embodiment, the GSH assay is that described in U.S. Pat. No. 8,697,391, which is incorporated herein by reference. The GSH assay provides a measurement of the efficiency of enzymes in six separate pathways that ensure that GSH can be repeatedly converted from reduced-to-oxidized-states. The measurement obtained using the methods and assays provided herein is in some cases referred to a “Chemotox” score. In certain embodiments, a Chemotox score is obtained from a single sample. In other embodiments, the Chemotox score is obtained from multiple samples and represents, for example, an average. The multiple samples can be obtained at different times during a patient's treatment.

Methods and assays for measuring GSH recycling activity are further described in WO 2018/071323 A1; Kutner et al. Support Care Cancer. 2017 February; 25(2):581-587. doi: 10.1007/s00520-016-3442-5; and McCourt et al. J Unexplored Med Data 2019; 4:6. doi: 10.20517/2572-8180.2019.01, all of which are incorporated herein by reference.

In one aspect, provided is a method of predicting or assessing the susceptibility of a subject to CIPN comprises assaying a biological sample containing RBC for a level of oxidative stress, wherein the samples obtained from a mammalian subject before and/or after administration of a chemotherapeutic agent. The method may be performed to assess risk of development of CIPN and/or predict severity of CIPN in the subject.

In certain embodiments, the indicator of oxidative stress is GSH recycling capacity. Accordingly, the subject's blood is obtained prior to treatment with a chemotherapeutic agent. In certain embodiments, the sample, e.g., a sample of whole blood, is obtained from a mammalian subject prior to administration of a chemotherapeutic agent. The sample is contacted with assay components that permit an assessment of the oxidative potential of the RBC in the sample, such as the GSH recycling dependent antioxidant activity of the blood cells. In one embodiment, the blood is tested for GSH recycling activity immediately post-draw. In other embodiments, the blood is tested for GSH recycling activity after storage. It is anticipated the GSH recycling activity of the blood remains stable for hours to days following draw.

In certain embodiments, the method involves contacting the sample with HEDS and further comprise the steps above described for the GSH assay, i.e., quantifying the amount of ME released by the sample treated with the HEDS and spectophotometrically measuring absorbance readings of ME; converting absorbance readings into ME concentrations; normalizing ME concentrations to total RBC count; and measuring the GSH recycling dependent antioxidant activity of intact RBC in said sample. These steps are performed to determine whether the subject's blood sample or biological sample is characterized as having a low or reduced (GSH) recycling dependent antioxidant activity. This method further permits assessment of whether a subject is susceptible to developing CIPN.

The methods are applicable to a subject having a cancer requiring treatment with a chemotherapeutic agent. The chemotherapeutic agent may be administered in any conventional method. In certain embodiments, the chemotherapeutic agent is administered intravenously. In certain embodiments, the chemotherapeutic agent is administered orally.

In one aspect, provided herein is a method of assessing susceptibility to chemotherapy-induced neuropathy (CIPN) in a patient administered a chemotherapeutic agent that includes (a) obtaining a measurement of glutathione (GSH) recycling activity in one or more biological samples containing red blood cells (RBC) obtained from the patient prior to administration of a chemotherapeutic agent; (b) obtaining a measurement of GSH recycling activity in one or more biological samples containing RBC obtained from the patient following treatment with the chemotherapeutic agent; and (c) comparing one or more measurements of GSH recycling activity in (a) with one or more measurements of GSH recycling activity in (b). In certain embodiments, a decrease in GSH recycling following treatment is predictive of increased susceptibility to CIPN. In certain embodiments, a decrease in GSH recycling following treatment is predictive of increased severity, frequency, and/or CIPN symptoms.

In one aspect, provided is a method for managing treatment of a patient with a chemotherapeutic agent and reducing the patient susceptibility to CIPN. The method includes (a) obtaining a measurement of GSH recycling activity in one or more biological samples containing red blood cells (RBC) obtained from the patient prior to administration of a chemotherapeutic agent; (b) obtaining a measurement of GSH recycling activity in one or more biological samples containing RBC obtained from the patient following treatment with the chemotherapeutic agent; (c) comparing one or more measurements of GSH recycling activity in (a) with one or more measurements of GSH recycling activity in (b); and (d) altering the subject's treatment with a chemotherapeutic agent, and/or administering a treatment for CIPN.

In certain embodiments, the method includes altering a treatment regimen during or after administration of a chemotherapeutic agent, when the subject's RBCs exhibit a reduced GSH recycling activity. In certain embodiments, when the subject's RBCs exhibit a reduced glutathione recycling capacity following treatment with the chemotherapeutic agent, the chemotherapeutic regimen is altered to decrease a subsequent dose or dosage of a chemotherapeutic agent delivered to the subject. In certain embodiments, the method includes discontinuing a regimen that includes one or more chemotherapeutic agent. In certain embodiments, the method includes starting treatment with a different or additional chemotherapeutic agent. Additional measurements of GSH recycling activity may be obtained following modification of the treatment regimen.

In certain embodiments, the method includes altering a treatment regimen during or after administration of a chemotherapeutic agent, when the subject's RBCs do not exhibit reduced GSH recycling activity. In certain embodiments, the chemotherapeutic regimen is altered to increase a subsequent dose or dosage of a chemotherapeutic agent delivered to the subject. In certain embodiments, the method includes continuing a regimen that includes one or more chemotherapeutic agent. In certain embodiments, the method includes starting treatment with a different or additional chemotherapeutic agent. Additional measurements of GSH recycling activity may be obtained following modification of the treatment regimen.

In one aspect, provided herein is a method for treating a patient with cancer that includes (a) obtaining a measurement of GSH recycling activity in one or more biological samples containing red blood cells (RBC) obtained from the patient prior to administration of a chemotherapeutic agent; (b) administering the chemotherapeutic agent; (c) obtaining a measurement of GSH recycling activity in one or more biological samples containing RBC obtained from the patient following treatment with the chemotherapeutic agent; and (d) altering the subject's treatment with a chemotherapeutic agent altering the subject's treatment with a chemotherapeutic agent, and/or administering a treatment for CIPN.

In certain embodiments, the method of treating the patient includes altering the dose or dosage of chemotherapeutic agent delivered to the patient when the subject's RBCs exhibit a reduced GSH recycling activity. In certain embodiments, when the subject's RBCs exhibit a reduced glutathione recycling capacity following treatment with the chemotherapeutic agent, the chemotherapeutic regimen is altered to decrease a subsequent dose or dosage of a chemotherapeutic agent delivered to the subject. In certain embodiments, the method includes discontinuing treatment of the subject with one or more chemotherapeutic agents. In certain embodiments, the method includes starting treatment with a different or additional chemotherapeutic agent.

In certain embodiments, the method of treating the patient includes altering the dose or dosage of chemotherapeutic agent delivered to the patient when the subject's RBCs do not exhibit reduced GSH recycling activity. In certain embodiments, the method includes increasing a subsequent dose or dosage of a chemotherapeutic agent delivered to the subject. In certain embodiments, the method includes continuing treatment of the subject with one or more chemotherapeutic agents. In certain embodiments, the method includes starting treatment with a different or additional chemotherapeutic agent.

In one aspect, provided herein is a method of reducing susceptibility to CIPN following treatment with a chemotherapeutic that includes (a) obtaining a measurement of glutathione (GSH) recycling activity in one or more biological samples containing red blood cells (RBC) obtained from the patient prior to administration of a chemotherapeutic agent; (b) obtaining a measurement of GSH recycling activity in one or more biological samples containing RBC obtained from the patient following treatment with the chemotherapeutic agent; (c) comparing one or more measurements of GSH recycling activity in (a) with one or more measurements of GSH recycling activity in (b); and (d) altering the patient's treatment depending upon a change in GSH recycling activity determined in (c) to reduce susceptibility to CIPN, and/or administering a treatment for CIPN.

In certain embodiments, the method of reducing susceptibility to CIPN in a subject includes altering the dose or dosage of a chemotherapeutic agent delivered to the subject when the subject's RBCs exhibit a reduced GSH recycling activity. In certain embodiments, when the subject's RBCs exhibit a reduced glutathione recycling capacity following treatment with the chemotherapeutic agent, a subsequent dose or dosage of a chemotherapeutic agent delivered to the subject is reduced. In certain embodiments, method of reducing susceptibility to CIPN in a subject includes discontinuing treatment of the subject with one or more chemotherapeutic agents. In certain embodiments, the method includes starting treatment with a different or additional chemotherapeutic agent. In certain embodiments, the method of reducing susceptibility to CIPN include one or treatments for CIPN, or the symptoms thereof.

In one aspect, provided herein is a method for assessing the ability of a treatment to alter susceptibility to CIPN in a subject that includes (a) obtaining a measurement of glutathione (GSH) recycling activity in one or more biological samples containing red blood cells (RBC) obtained from the subject prior to administration of a chemotherapeutic agent (b) obtaining a measurement of GSH recycling activity in one or more biological samples containing RBC obtained from the patient following treatment with the chemotherapeutic agent; (c) comparing one or more measurements of GSH recycling activity in (a) with one or more measurements of GSH recycling activity in (b); and (d) administering the treatment for CIPN to the subject.

In certain embodiments, the method includes administering the chemotherapeutic agent in combination with the treatment for CIPN. In certain embodiments, the CIPN treatment is administered before administering the chemotherapeutic agent. In certain embodiments, the CIPN treatment is administered following administration of the chemotherapeutic agent. In certain embodiments, the method includes obtaining a measurement of glutathione (GSH) recycling activity in one or more biological samples containing red blood cells (RBC) obtained from the subject prior to administration of the chemotherapeutic agent and the treatment for CIPN. In certain embodiments, the method includes obtaining a measurement of glutathione (GSH) recycling activity in one or more biological samples containing red blood cells (RBC) obtained from the subject following administration of the chemotherapeutic agent and the treatment for CIPN. In certain embodiments, the method includes obtaining a measurement of glutathione (GSH) recycling activity in one or more biological samples containing red blood cells (RBC) obtained from the subject following administration of the chemotherapeutic agent and before treatment for CIPN. In certain embodiments, the method includes obtaining a measurement of glutathione (GSH) recycling activity in one or more biological samples containing red blood cells (RBC) obtained from the subject following treatment for CIPN and prior to administration of the chemotherapeutic agent.

The methods provided herein are intended to be used in a clinical setting, including evaluating a patient's treatment during a regimen of chemotherapy. The regimen may be over a period of days, weeks, or years and involve multiple treatments or administrations of a chemotherapeutic agent. In certain embodiments, a subject is administered at least at least 2, at least 3, at least 4, at least 5, at least 6, at least 7, at least 8, at least 9, at least 10, at least 11, at least 12, at least 13, at least 14, at least 15, or at least 16 treatments with a chemotherapeutic agent as part of a treatment regimen. In certain embodiments, a measurement is obtained from a sample acquired prior to the start of treatment (including on the day of treatment prior to administration of chemotherapy). In some instances, this measurement is referred to as a baseline measurement, a pre-treatment measurement, or a “pre1” measurement. In addition, one or more measurement can be obtained from samples acquired at times following the first administration of a chemotherapeutic agent but prior to the second administration of a chemotherapeutic agent (including the day of the second administration), or following the second administration of a chemotherapeutic agent but prior to the third administration of a chemotherapeutic agent (including the day of the third administration), and so on.

In one aspect, provided herein is a method of assessing susceptibility to or severity of chemotherapy-induced neuropathy (CIPN) in a subject administered a chemotherapeutic agent that includes (a) obtaining a baseline measurement of glutathione (GSH) recycling activity in one or more biological samples containing red blood cells (RBC) obtained from the subject prior to a first administration of a chemotherapeutic agent; (b) obtaining a first measurement of GSH recycling activity in one or more biological samples containing RBC obtained from the subject following a first administration of the chemotherapeutic agent, but prior to a second administration of the chemotherapeutic agent; (c) obtaining a second measurement of GSH recycling activity in one or more biological samples containing RBC obtained from the subject following the second administration of the chemotherapeutic agent, but prior to a third administration of the chemotherapeutic agent; (d) obtaining a third measurement of GSH recycling activity in one or more biological samples containing RBC obtained from the subject following the third administration of the chemotherapeutic agent, but prior to a fourth administration of the chemotherapeutic agent; (e) obtaining an average of the baseline measurement, the first measurement, the second measurement, and the third measurement. In certain embodiments, an average of 1.85 or less is indicative of susceptibility to or severity of CIPN. In certain embodiments, an average of 1.50 or less, 1.55 or less, 1.65 or less, 1.70 or less, 1.75 or less, 1.80 or less, 1.90 or less, 1.95 or less, or 2.00 or less is indicative of susceptibility to or severity of CIPN.

In another aspect, provided herein is a method of assessing susceptibility to or severity of chemotherapy-induced neuropathy (CIPN) in a subject administered a chemotherapeutic agent that includes (a) obtaining a first measurement of GSH recycling activity in one or more biological samples containing RBC obtained from the subject following a first administration of the chemotherapeutic agent, but prior to a second administration of the chemotherapeutic agent; (b) obtaining a second measurement of GSH recycling activity in one or more biological samples containing RBC obtained from the subject following the third administration of the chemotherapeutic agent, but prior to a fourth administration of the chemotherapeutic agent; (c) obtaining an average of the baseline measurement, the first measurement, and the second measurement. In certain embodiments, an average of 1.8 or less is indicative of susceptibility to or severity of CIPN. In certain embodiments, an average of 1.50 or less, 1.55 or less, 1.65 or less, 1.70 or less, 1.75 or less, 1.85 or less, 1.90 or less, 1.95 or less, or 2.00 or less is indicative of susceptibility to or severity of CIPN.

In another aspect, provided herein is a method assessing susceptibility to or predicting severity of chemotherapy-induced neuropathy (CIPN) in a subject administered a chemotherapeutic agent, the method that includes (a) obtaining a baseline measurement of glutathione (GSH) recycling activity in one or more biological samples containing red blood cells (RBC) obtained from the subject prior to a first administration of a chemotherapeutic agent; (b) obtaining (i) a measurement of GSH recycling activity in one or more biological samples containing RBC obtained from the subject following a first administration of the chemotherapeutic agent, but prior to a second administration of the chemotherapeutic agent; (ii) a measurement of GSH recycling activity in one or more biological samples containing RBC obtained from the subject following a second administration of the chemotherapeutic agent, but prior to a third administration of the chemotherapeutic agent; or (iii) obtaining a measurement of GSH recycling activity in one or more biological samples containing RBC obtained from the subject following a third administration of the chemotherapeutic agent, but prior to a fourth administration of the chemotherapeutic agent; wherein a decrease in GSH recycling activity is predictive of increased susceptibility to or severity of CIPN.

In another aspect, provided herein is a method of reducing susceptibility to CIPN following treatment with a chemotherapeutic agent that includes (a) obtaining a baseline measurement of glutathione (GSH) recycling activity in one or more biological samples containing red blood cells (RBC) obtained from the subject prior to a first administration of a chemotherapeutic agent; (b) obtaining a first measurement of GSH recycling activity in one or more biological samples containing RBC obtained from the subject following a first administration of the chemotherapeutic agent, but prior to a second administration of the chemotherapeutic agent; (c) obtaining a second measurement of GSH recycling activity in one or more biological samples containing RBC obtained from the subject following the second administration of the chemotherapeutic agent, but prior to a third administration of the chemotherapeutic agent; (d) obtaining a third measurement of GSH recycling activity in one or more biological samples containing RBC obtained from the subject following the third administration of the chemotherapeutic agent, but prior to a fourth administration of the chemotherapeutic agent; (e) obtaining an average of the baseline measurement, the first measurement, the second measurement, and the third measurement; and (f) altering the subject's treatment regimen to reduce susceptibility to or severity of chemotherapy-induced neuropathy (CIPN), and/or administering a treatment for CIPN. In certain embodiments, an average of 1.85 or less is indicative of susceptibility to or severity of CIPN. In certain embodiments, an average of 1.50 or less, 1.55 or less, 1.65 or less, 1.70 or less, 1.75 or less, 1.80 or less, 1.90 or less, 1.95 or less, or 2.00 or less is indicative of susceptibility to or severity of CIPN.

In another aspect, provided herein is a method of reducing susceptibility to CIPN following treatment with a chemotherapeutic agent that includes (a) obtaining a first measurement of GSH recycling activity in one or more biological samples containing RBC obtained from the subject following a first administration of the chemotherapeutic agent, but prior to a second administration of the chemotherapeutic agent; (b) obtaining a second measurement of GSH recycling activity in one or more biological samples containing RBC obtained from the subject following the third administration of the chemotherapeutic agent, but prior to a fourth administration of the chemotherapeutic agent; (c) obtaining an average of the first measurement and the second measurement; and (d) altering the subject's treatment regimen to reduce susceptibility to or severity of chemotherapy-induced neuropathy (CIPN), and/or administering a treatment for CIPN. In certain embodiments, an average of 1.8 or less is indicative of susceptibility to or severity of CIPN. In certain embodiments, an average of 1.50 or less, 1.55 or less, 1.65 or less, 1.70 or less, 1.75 or less, 1.85 or less, 1.90 or less, 1.95 or less, or 2.00 or less is indicative of susceptibility to or severity of CIPN.

In another aspect, provided herein is a method of reducing susceptibility to CIPN following treatment with a chemotherapeutic agent that includes (a) obtaining a baseline measurement of glutathione (GSH) recycling activity in one or more biological samples containing red blood cells (RBC) obtained from the subject prior to a first administration of a chemotherapeutic agent; (b) obtaining (i) a measurement of GSH recycling activity in one or more biological samples containing RBC obtained from the subject following a first administration of the chemotherapeutic agent, but prior to a second administration of the chemotherapeutic agent; (ii) a measurement of GSH recycling activity in one or more biological samples containing RBC obtained from the subject following a second administration of the chemotherapeutic agent, but prior to a third administration of the chemotherapeutic agent; or (iii) obtaining a measurement of GSH recycling activity in one or more biological samples containing RBC obtained from the subject following a third administration of the chemotherapeutic agent, but prior to a fourth administration of the chemotherapeutic agent; wherein a decrease in GSH recycling activity is predictive of increased susceptibility to or severity of CIPN; and (c) altering the subject's treatment regimen to reduce susceptibility to or severity of chemotherapy-induced neuropathy (CIPN), and/or administering a treatment for CIPN.

In certain embodiments, altering a subject's treatment inducing reducing a subsequent dose or dosage of the chemotherapeutic agent. For example, a patient's scheduled treatment regimen can be altered based on the measured GSH recycling activity. In certain embodiments, altering the subject's treatment includes treatment with a different chemotherapeutic agent. In certain embodiments, altering the subject's treatment includes administering an alternative or additional chemotherapeutic agent. In further embodiments, altering the subject's treatment comprises discontinuing administration of a chemotherapeutic agent. In certain embodiments, altering the subject's treatment includes treatment with a different chemotherapeutic agent.

In one aspect, provided herein is a method for assessing the ability of a treatment to alter susceptibility to or severity of chemotherapy-induced neuropathy (CIPN) in a subject that includes (a) obtaining a baseline measurement of glutathione (GSH) recycling activity in one or more biological samples containing red blood cells (RBC) obtained from the subject prior to a first administration of a chemotherapeutic agent; (b) obtaining a first measurement of GSH recycling activity in one or more biological samples containing RBC obtained from the subject following a first administration of the chemotherapeutic agent, but prior to a second administration of the chemotherapeutic agent; (c) obtaining a second measurement of GSH recycling activity in one or more biological samples containing RBC obtained from the subject following the second administration of the chemotherapeutic agent, but prior to a third administration of the chemotherapeutic agent; (d) obtaining a third measurement of GSH recycling activity in one or more biological samples containing RBC obtained from the subject following the third administration of the chemotherapeutic agent, but prior to a fourth administration of the chemotherapeutic agent; (e) obtaining an average of the baseline measurement, the first measurement, the second measurement, and the third measurement; and (f) administering the treatment for CIPN to the subject. In certain embodiments, an average of 1.85 or less is indicative of susceptibility to or severity of CIPN. In certain embodiments, an average of 1.50 or less, 1.55 or less, 1.65 or less, 1.70 or less, 1.75 or less, 1.80 or less, 1.90 or less, 1.95 or less, or 2.00 or less is indicative of susceptibility to or severity of CIPN.

In one aspect, provided herein is a method for assessing the ability of a treatment to alter susceptibility to or severity of chemotherapy-induced neuropathy (CIPN) in a subject that includes (a) obtaining a first measurement of GSH recycling activity in one or more biological samples containing RBC obtained from the subject following a first administration of the chemotherapeutic agent, but prior to a second administration of the chemotherapeutic agent; (b) obtaining a second measurement of GSH recycling activity in one or more biological samples containing RBC obtained from the subject following the third administration of the chemotherapeutic agent, but prior to a fourth administration of the chemotherapeutic agent; (c) obtaining an average of the baseline measurement, the first measurement, and the second administration; and (d) administering the treatment for CIPN to the subject. In certain embodiments, an average of 1.8 or less is indicative of susceptibility to or severity of CIPN. In certain embodiments, an average of 1.50 or less, 1.55 or less, 1.65 or less, 1.70 or less, 1.75 or less, 1.85 or less, 1.90 or less, 1.95 or less, or 2.00 or less is indicative of susceptibility to or severity of CIPN.

In one aspect, provided herein is a method for assessing the ability of a treatment to alter susceptibility to or severity of chemotherapy-induced neuropathy (CIPN) in a subject that includes: (a) obtaining a baseline measurement of glutathione (GSH) recycling activity in one or more biological samples containing red blood cells (RBC) obtained from the subject prior to a first administration of a chemotherapeutic agent; (b) obtaining (i) a measurement of GSH recycling activity in one or more biological samples containing RBC obtained from the subject following a first administration of the chemotherapeutic agent, but prior to a second administration of the chemotherapeutic agent; (ii) a measurement of GSH recycling activity in one or more biological samples containing RBC obtained from the subject following a second administration of the chemotherapeutic agent, but prior to a third administration of the chemotherapeutic agent; or (iii) obtaining a measurement of GSH recycling activity in one or more biological samples containing RBC obtained from the subject following a third administration of the chemotherapeutic agent, but prior to a fourth administration of the chemotherapeutic agent; wherein a decrease in GSH recycling activity is predictive of increased susceptibility to or severity of CIPN; and (c) administering the treatment for CIPN to the subject.

In certain embodiments, the method provided is predictive of increased susceptibility to Grade 0 CIPN, Grade 1 CIPN, Grade 2 CIPN, or Grade 3 CIPN. The methods can be predictive of CIPN that develops during a treatment regimen or after a treatment regimen. In certain embodiments, the method predicts the development of CIPN at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 week, at least 14 weeks, at least 15 weeks, at least 16 weeks, at least 17 weeks, at least 18 weeks, at least 19 weeks, at least 20 weeks, at least 3 months, at least 6 months, or at least a year prior to experiencing symptoms of CIPN or severe CIPN.

A change in GSH recycling activity for an individual can be measure using a measurement obtained prior to treatment and during treatment or measurements obtained during treatment (e.g., following various rounds of treatment). In certain embodiments, a decrease in GSH recycling activity is a percentage decrease calculated as (measurement obtained at a time during treatment−baseline)/baseline). A decrease in GSH recycling that is predictive of increased susceptibility to or severity of CIPN can be at least 5%, at least 10%, at least 15%, at least 20%, at least 25%, at least 30%, at least 35%, at least 40%, at least 45%, or at least 50%.

In certain embodiments, the methods provided are directed to a certain age or gender of patient. In certain embodiments, the patient is male. In certain embodiments, the patient in female. In certain embodiments the patient is 25-100 years old, 25-50 years old, 25-75 years old, 50-75 years old, or 50-100 years old.

As used herein, the term “treatment” or “treating” refers to composition(s) and/or method(s) for the purposes of amelioration of one or more symptoms of a disease or condition. For example, treatment for CIPN can include administering a composition to alleviate neuropathy or hyperalgesia, included the prevention or reduction the frequency, severity, and/or duration of symptoms, such as pain in the extremities. Treatment can thus include one or more of reducing onset or progression of CIPN, preventing CIPN, reducing the frequency and/or severity of CIPN symptoms (e.g., pain the extremities), retarding the progression of CIPN, and/or delaying progression of CIP. Treatments for CIPN include, without limitation, oral pain medications, antidepressants, lidocaine patches, menthol creams, a serotonin-norepinephrine reuptake inhibitor (SNRI) (e.g., duloxetine), tricyclic antidepressants (TCAs), anticonvulsants, compounded topical products, nonsteroidal anti-inflammatory drugs (NSAIDs), and opioid therapy, physical therapy, and occupational therapy. In certain embodiments, the treatment reduces the frequency and/or severity of symptoms in a treated subject relative to an untreated control subject. As used herein, a treatment for CIPN includes compositions that are under investigation for their potential as a treatment of CIPN (i.e., test compounds).

In certain embodiments, the treatment for CIPN includes administering a “sodium channel blocker,” which is a chemical compound that binds selectively to a sodium channel and thereby deactivates the sodium channel. In particular, sodium channel blockers include compounds that bind to the SS1 or SS2 extracellular domains of an alpha subunit of a sodium channel. Sodium channel blocking compounds that bind to the SS1 or SS2 subunit of a sodium channel, particularly tetrodotoxin and saxitoxin, are found to possess similar pharmaceutical activity (See, e.g., U.S. Pat. No. 6,407,088, which is incorporated herein by reference).

In certain embodiments, the treatment for CIPN includes administering calcium/magnesium supplementation to the subject. In certain embodiments, the treatment for CIPN includes a calpain inhibitor.

In certain embodiments, the treatment for CIPN includes administering a composition containing one or more phytocannabinoids, or synthetic derivates thereof. In certain embodiment, the treatment for CIPN includes administering a composition that includes cannabidiol (CBD).

As used herein, the phrase low glutathione (GSH) recycling dependent antioxidant activity in the biological sample (i.e., high oxidative stress) is used to indicate a predisposition to CIPN. In certain embodiment, a low GSH recycling dependent antioxidant activity score is ≤1.0. As used herein, the phrase high glutathione recycling dependent antioxidant activity in the biological sample (i.e., low oxidative stress) is used to indicate an increased risk of CIPN. In certain embodiments, a high GSH recycling dependent antioxidant activity score is >1.0.

As described herein, the present inventors have identified methods that rely, for example, on a comparison of one or more measurements of GSH recycling activity in a patient sample obtained before administering a chemotherapeutic agent with one or more measurements of GSH recycling activity in a patient sample obtained after administering a chemotherapeutic agent. In certain embodiments, the method includes obtaining measurements from more than one day or over the course of weeks or months before and/or after administering a chemotherapeutic agent. In certain embodiments, the method includes a comparison of an average of measurements of GSH recycling activity obtained on a single day before and/or after administering a chemotherapeutic agent. In certain embodiments, the methods include obtaining an average value for GSH recycling activity based on measurements obtained on the same day or over the course of days or weeks before administering a chemotherapeutic agent to the subject. In certain embodiments, the methods include obtaining an average value for GSH recycling activity based on measurements obtained on the same day or over the course of days or weeks after administering a chemotherapeutic agent to the subject. Thus, the “measurement” of GSH recycling activity can refer to an average of measurements obtained from a collection of samples obtained, for example, before treatment with a chemotherapeutic agent or after treatment with a chemotherapeutic agent.

In certain embodiments, the methods include obtaining one or more measurements of GSH recycling activity in one or more biological samples obtained from the subject less than 12 hours, less than 24 hours, or less than 36 hours before treatment with a chemotherapeutic agent. In certain embodiments, the methods include obtaining one or more measurements of GSH recycling activity in one or more biological samples obtained from the subject less than 12 hours, less than 24 hours, or less than 36 hours following treatment with a chemotherapeutic agent.

A reduction in or reduced GSH recycling activity following treatment of a subject with a chemotherapeutic agent is associated with an increased risk for CIPN. In certain embodiments, the methods include identifying a reduction in GSH recycling activity where there is an about 10% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 85% or greater, about 90% or greater, or at least about 95% or greater reduction, or any amount of reduction in between the specifically recited percentages, as compared to the pre-treatment measurement.

In certain embodiments, the methods include identifying the absence of a reduction in GSH recycling activity or, when compared to a measurement obtained from a pre-treatment sample, GSH recycling activity is relatively unchanged post treatment. In certain embodiments, the methods include identifying the absence of reduction in GSH recycling activity (or unchanged) where the measurement obtained post-treatment deviates less than about 5%, less than about 10%, less than about 15%, less than about 20%, less than about 25%, less than about 30%, less than about 35%, less than about 40%, less than about 45%, less than about 50%, or any amount of reduction in between the specifically recited percentages, from the pre-treatment measurement(s).

In certain embodiments, the methods include diagnosing CIPN or assessing severity of CIPN in a subject. A diagnosis of CIPN can be based on the results of both subjective and/or objective assessment methods. In certain embodiment, the methods include subjective evaluation such as patient accounts, prior and current treatments (both chemotherapeutic and nonchemotherapeutic agents), and social history. In certain embodiments, the methods include objective evaluation strategies such as a physical examination, laboratory tests, standardized questionnaires, electrodiagnostic studies, and nerve biopsy. The National Comprehensive Cancer Network (NCCN) details the most common physician-based grading scales used to assess the severity of CIPN. These grading scales include the Ajani Sensory, Eastern Cooperative Oncology Group (ECOG), National Cancer Institute-Common Terminology Criteria for Adverse Events (NCI-CTCAE), and World Health Organization (WHO) systems, which grade CIPN severity on a scale of 0 (normal) to 5 (death).

In still another aspect, a method of determining an individualized chemotherapeutic regimen for an individual human subject with cancer includes performing an assay to evaluate the subject's blood sample's glutathione recycling dependent antioxidant activity and efficiency for scavenging free radicals prior to initiating chemotherapy is provided.

As demonstrated in the examples below, the redox assay capable of measuring GSH recycling in the blood was used to compare a patient's intrinsic ability to recycle GSH, before receiving chemotherapy with their later susceptibility to CINV after receiving treatment. The examples focused on platinum-based therapies. However, the supporting data shown in the examples below can be extended to broader patient demographics. The examples describe preliminary results from showing that a reduced ability to recycle GSH in the blood provides an objective indicator of the development of CIPN.

### Assay

As described above, methods for assessing the recycling capacity of the antioxidant glutathione in a sample are known in the art. The present inventors developed methods that improve upon previous assays, including OxPhos™ Cell Survival Kit (cat. no. KLD-02, Rockland Inc.) that rely on the conversion of hydroxyethyl disulfide (HEDS) into mercaptoethanol (ME) through a bioreduction mechanism (See, also, U.S. Pat. No. 8,697,391, which is incorporated herein by reference). The improved methods facilitate processing of large numbers of samples (e.g., in a 96-well plate format) and produce accurate measurements when using an automated plate reader typically found in CLIA certified clinical laboratories. Notably, certain methods do not require processing samples with a sulfosalicyclic acid (SSA) buffer.

In one aspect, provided herein is an assay for measuring glutathione (GSH) recycling capacity in a sample containing red blood cells (RBC) that includes (a) combining the sample with a solution containing hydroxy-ethyl-disulfide (HEDS) to obtain a first volume, and incubating said first volume to allow a substantial fraction of RBC from the sample to sediment; (b) obtaining an aliquot of the first volume, substantially free of RBC, and admixing said aliquot of the first volume with a solution containing magnetic nanobeads to obtain a second volume; (c) subjecting the second volume, or an aliquot thereof, to a magnetic field; (d) obtaining a supernatant from the second volume, and combining the supernatant, or aliquot thereof, with a solution containing 5,5′-Disulfanediylbis(2-nitrobenzoic acid) (DNTB) to obtain a third volume; and (e) subjecting the third volume, or aliquot thereof, to spectrophotometric analysis to obtain an absorbance reading, wherein the absorbance measured in (e) is indicative of GSH recycling capacity in the sample containing RBC.

In another aspect, provided herein is an assay for measuring GSH recycling capacity in a collection of samples containing RBC that includes (a) combining each of the samples with a solution containing HEDS to obtain a collection of first volumes, and incubating said collection of first volumes to allow a substantial fraction of RBCs from the sample to sediment; (b) obtaining an aliquot of each of the said first volumes, substantially free of RBCs, and admixing each aliquot with a solution containing magnetic nanobeads to obtain a collection of second volumes; (c) subjecting the collection of second volumes, or aliquots thereof, to a magnetic field; (d) obtaining a collection of supernatants from the second volumes, and combining each of the supernatants, or aliquots thereof, with a solution containing 5,5′-Disulfanediylbis(2-nitrobenzoic acid) (DNTB) to obtain a collection of third volumes; and (e) subjecting the collection of third volumes, or aliquot thereof, to spectrophotometric analysis to obtain a series of absorbance readings, wherein the absorbances measured in (e) are indicative of GSH recycling capacity in the samples containing RBC.

In another aspect, provided herein is an assay for measuring GSH recycling capacity in a collection of samples containing RBC that includes (a) combining each of the samples with a solution containing HEDS to obtain a collection of first volumes, and incubating said collection of first volumes; (b) centrifuging the samples to remove RBCs and debris from suspension; (c) obtaining an aliquot of each of the said first volumes, substantially free of RBC and debris, and admixing each aliquot with trichloroacetic acid (TCA) to obtain a collection of second volumes; (d) subjecting the collection of second volumes, or aliquots thereof, to centrifugation; (e) obtaining a collection of supernatants from the second volumes, and combining each of the supernatants, or aliquots thereof, with a solution containing 5,5′-Disulfanediylbis(2-nitrobenzoic acid) (DNTB) to obtain a collection of third volumes; and (f) subjecting the collection of third volumes, or aliquots thereof, to spectrophotometric analysis to obtain a series of absorbance readings, wherein the absorbance readings obtained in (e) are indicative of GSH recycling capacity in the sample containing RBC.

As described in Example 3, the inventors have developed an assay that can be performed using an 8-well strip or a series of 8-well strips. In certain embodiments of the assay, each 8-well strip is allocated to each sample. The design of the assay facilitates processing of samples and improves accuracy of the measurements as compared to available assays. The 8-well strips are readily available and are designed to fit into a frame that holds up to 12 strips (e.g., see design of J G Finneran and Porvair Sciences, Model #208107, 96 well plate, 8-well strips on 12×8 frame). The 8-well strips are available in V-bottom, U-bottom, and flat-bottom depending on application.

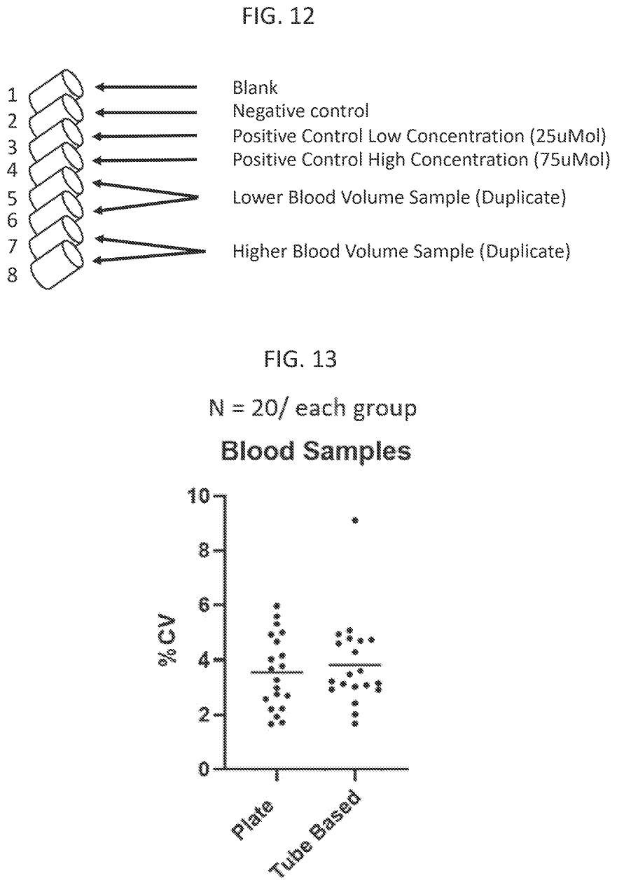

As depicted, for example, in FIG. 12 the wells of a 8-well strip allow for one or more of a blank, negative, positive control in addition to dilutions of an RBC-containing sample. In certain embodiments, the wells are in duplicate to control for accuracy. The arrangement of sample volumes in an 8-well strip facilitates the transfer of samples and their organization at various steps of the assay, including transferring supernatants following a clarification step (e.g, centrifugation).

As described herein, the inventors have identified a useful positive control, L-Cysteine hydrochloride monohydrate (LCHM), that can be included in an assay for measuring GSH recycling activity. L-Cysteine hydrochloride monohydrate (LCHM) is readily available through various providers (e.g., Alfa Aesar, CAS 7048-04-6) in lyophilized form or dissolved in a buffer, for example. The positive control can be included in the assay at one or more concentrations. In certain embodiments, LCHM is included in solution at a concentration of about 25 μMol, about 50 μMol, and/or about 25 μMol to about 50 μMol. LCHM can be dissolved in, for example, UltraPure Water (Cayman Chemicals) at a low pH.

In certain embodiments, the first volume (or each volume in a collection of first volumes) is about 250 μl or less, about 200 μl or less, about 150 μl or less, about 100 μl or less, about 75 μl or less, about 50 μl or less, or about 25 μl or less. In certain embodiments, the second volume (or each volume in a collection of second volumes) is about 250 μl or less, about 200 μl or less, about 150 μl or less, about 100 μl or less, about 75 μl or less, about 50 μl or less, or about 25 μl or less. In certain embodiments, the third volume (or each volume in a collection of third volumes) is about 350 μl or less, about 300 μl or less, about 250 μl or less, about 200 μl or less, about 150 μl or less, about 100 μl or less, about 75 μl or less, about 50 μl or less, or about 25 μl or less. In certain embodiments, the methods include at least about 5, at least about 10, at least about 20, at least about 50, at least about 100, at least about 200, at least about 250, at least about 300, or at least about 350 first volumes, second volumes, and/or third volumes.

In certain embodiments, step (a) of the described methods includes incubating the sample(s) for at least about 15 minutes, at least about 30 minutes, at least about 1 hour, at least about 1.5 hours, or at least about 2 hours. The incubation period allows for the conversion of hydroxyethyl disulfide (HEDS) into mercaptoethanol (ME) as well as the sedimentation of RBC. In certain embodiments, step (a) includes shaking the sample for all or part of the incubation time, e.g. at 700 rpm. In a certain embodiment, the first volumes are incubated in a V-bottom microplate. In certain embodiments, the plate is a 96-well microplate. In certain embodiments, the plate is a 384-well microplate. In certain step (a) includes incubating the sample volumes at or at about 21° C. Where incubation is not performed at 21° C., the following formula can be utilized to obtain an adjusted final GSH activity measurement: Chemotox_{FINAL}=(Chemotox_{INITIAL}−(0.159*T_{ACTUAL}))+(0.159*₂₁).

In certain embodiments, the magnetic nanobeads of the described methods are glutathione magnetic agarose beads (See, e.g. Pierce™ Glutathione Magnetic Agarose Beads; Catalog number: 78601). In certain embodiments, the nanobeads are made of silica (SiO₂) and maghemite (γ-Fe₂O₃). Suitable nanobeads made of silica (SiO₂) and maghemite (γ-Fe₂O₃), or similar, can be produced according to the methods described in Stöber, Werner; Fink, Arthur; Bohn, Ernst (January 1968). “Controlled growth of monodisperse silica spheres in the micron size range”. Journal of Colloid and Interface Science., which is incorporated herein by reference.

Magnetic separation can be achieved using convention means available to those of skill in the art. In certain embodiments, the method includes contacting a plate or sample-containing wells that contain the sample with a magnetic separation device (e.g. a Dexter LifeSep® biomagnetic separator tray; Dexter Magentic Technologies). Other suitable magnetic separation devices are known in the art.

As previously described, the extracellular ME in a biological sample can be measured by a 5,5-dithiobis 2-nitrobenzoic acid (DTNB) assay (see, e.g., Ayene et al. (2002) J. Biol. Chem., 277: 9929-35). For example, extracellular media is mixed with DTNB and then the O.D. may be measured at 412 nm. The concentration of ME may be calculated using an extinction coefficient of 1.36×10⁴ for reduced DTNB. In certain embodiments, measurements of absorbance in the described methods are obtained using an automated plate reader. In certain embodiments, step measuring absorbance is performed on samples in a flat-bottom plate. In certain embodiments, the plate is a 96-well microplate. In certain embodiments, the plate is a 384-well microplate. In certain embodiments, the plate is an optical bottom microplate. In certain embodiments, measuring absorbance is performed on one or more 8-well strips in a frame capable of holding up to twelve 8-well strips. In certain embodiments, the method includes comparing the amount of absorbance from a test sample with at least one standard. For example, the standard may be the amount from a biological sample (e.g., from the subject). In certain embodiments, the standard is a solution containing hydroxy-ethyl-disulfide (HEDS) and L-Cysteine hydrochloride monohydrate (LCHM).

### Kits

Kits for performing the methods described herein are also provided.

In certain embodiments, the kit includes HEDS and nanobeads. In further embodiment, the kit includes HEDS, nanobeads, and DTNB. In certain embodiments, the one or more of the HEDS, nanobeads, and DTNB is contained in a composition that includes a carrier. In certain embodiments, the kit further includes one or more microtiter plates. In certain embodiments, the kit includes a V-bottom microplate.

In another embodiment, the kit includes hydroxy-ethyl-disulfide (HEDS) and L-Cysteine hydrochloride monohydrate (LCHM) as a positive control. In certain embodiments, the LCHM is lyophilized. In other embodiments, the LCHM is in a solution at a concentration of about 25 μMol, about 50 μMol, and/or about 25 μMol to about 50 μMol. In certain embodiments, the also includes one or more of DTNB (Ellman's Reagent), V-bottom 8-well strips, flat-bottom 8-well strips, a frame capable of holding up to twelve 8-well strips, trichloroacetic acid (TCA), and a glutathione buffer.

The invention is now described with reference to the following examples. These examples are provided for the purpose of illustration only and the invention should in no way be construed as being limited to these examples but rather should be construed to encompass any and all variations that become evident as a result of the teachings provided herein.

### EXAMPLES

### Example 1: Measuring GSH Recycling Activity to Assess Susceptibility to CIPN and Predicting Severity of CIPN

Vinca alkaloids, such as vincristine, are important anticancer agents that are mainly employed in the treatment of hematological cancers. The principal mechanism for their antineoplastic activity is microtubule disruption. However, these agents also cause damage to mitochondria which leads to oxidative stress and production of reactive oxygen species (ROS). Other chemotherapeutic drugs that are suggested to cause peripheral neuropathy by this mechanism, i.e., increased oxidative stress, include taxanes and platinum compounds. Oxidative damage to peripheral neurons can cause damage to myelin sheath, mitochondrial proteins, and other antioxidant enzymes, resulting in hyperexcitability of peripheral neurons. This nerve damage results in the commonly seen dose-limiting neurological side effect chemotherapy-induced peripheral neuropathy (CIPN).

We hypothesized that chemotherapeutic agents with similar effects on mitochondria (vinca alkaloids, taxanes and platinum compounds) would produce a specific pattern of glutathione recycling in patients prone to CIPN.

Methods: Patients who had given written consent to participate in this exploratory single-centered, prospective IRB approved study contributed a blood sample prior to each treatment cycle. A baseline/pre-treatment sample was obtained prior to beginning the treatment regimen. Additional samples were obtained on the day of each treatment prior to administration of any chemotherapy. At each visit, the Rotterdam Symptoms Check-List (RSCL) was filled out and reported symptoms confirmed by comparison to notes in medical records.

Whole blood was analyzed for glutathione recycling capacity using the bioactive probe hydroxyethyldisulfide (HEDS) and incubated at room temperature for two hours with gentle mixing. Prior to spectrophotometric determination, blood cells and proteinaceous thiols were removed from the sample by acid precipitation and centrifugation. The final spectrophotometric reading was be converted into ME produced using the conversion factor provided in the OxPhos assay kit (Rockland Inc) to obtain a “chemotox” score. Recycling capacity (Chemotox score) was compared to self-reported grade of CIPN. “Severe CIPN” refers to patients who self-reported Grade 3 CIPN for 3 or more consecutive treatment visits. FIG. 1 provides a table showing guidelines for grading nervous system disorders, including CIPN.

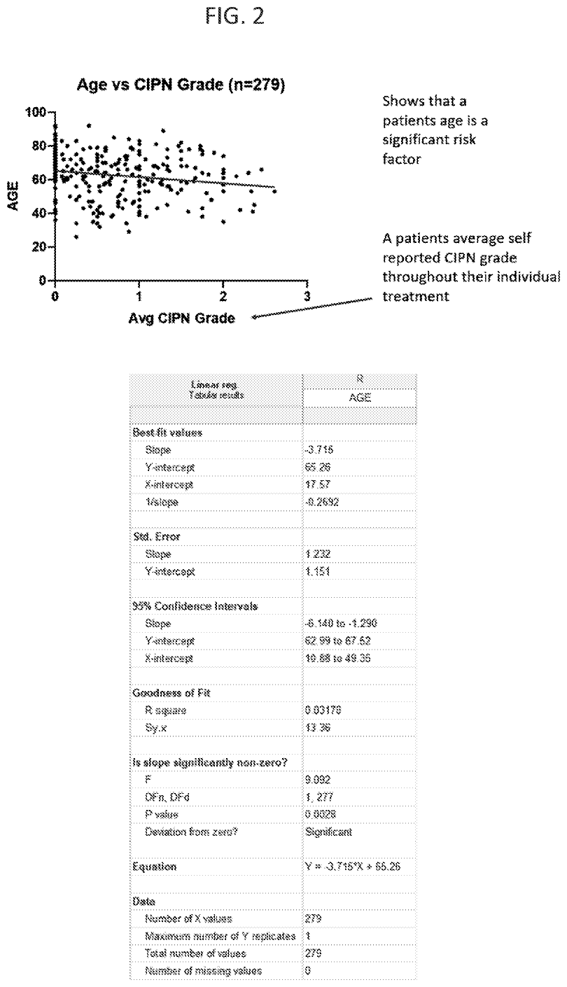

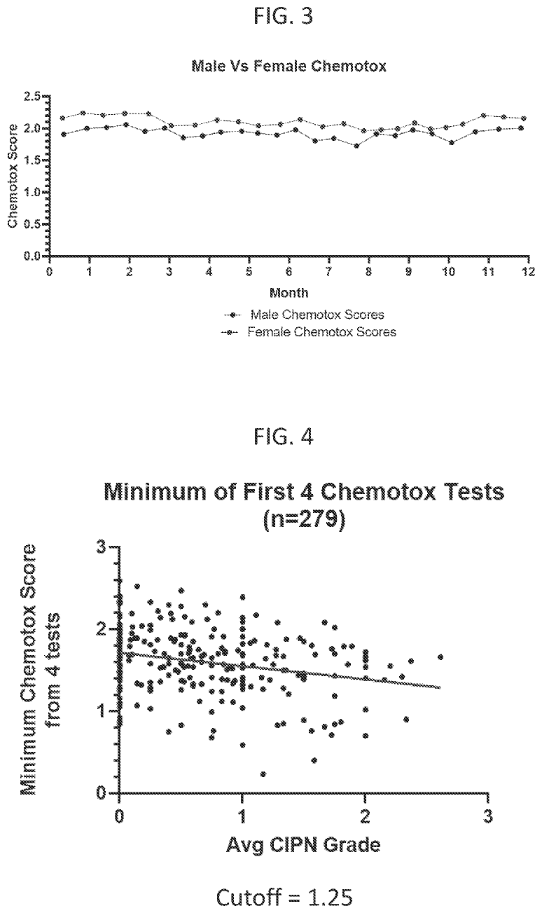

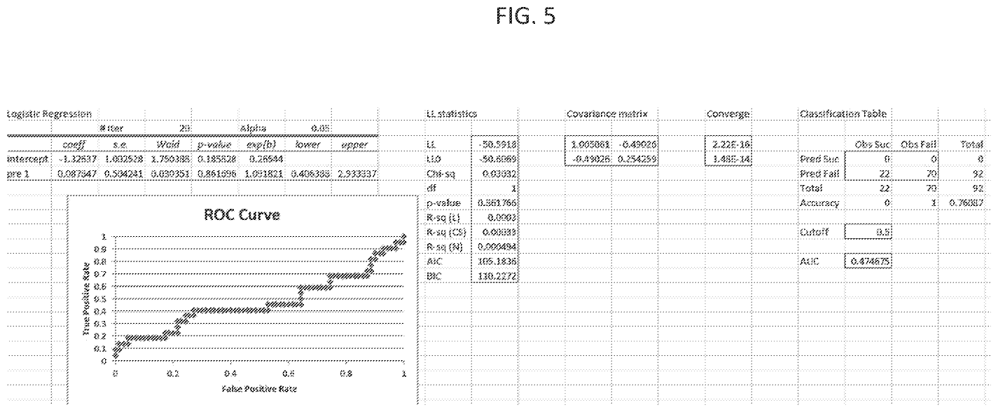

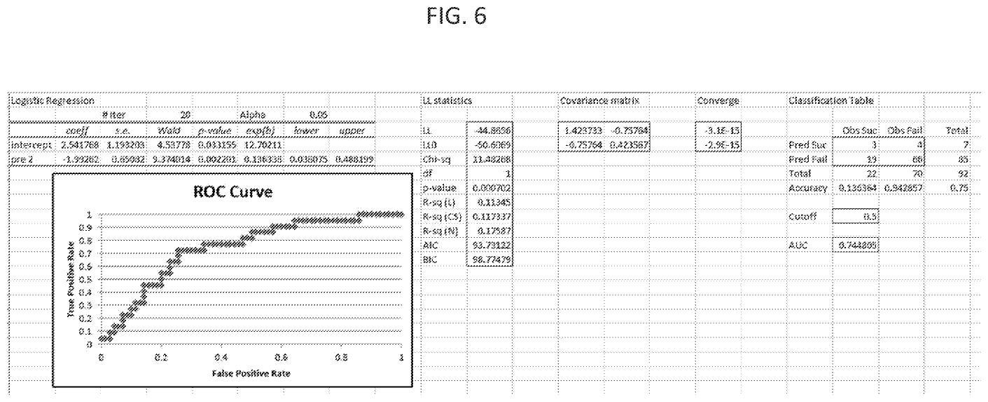

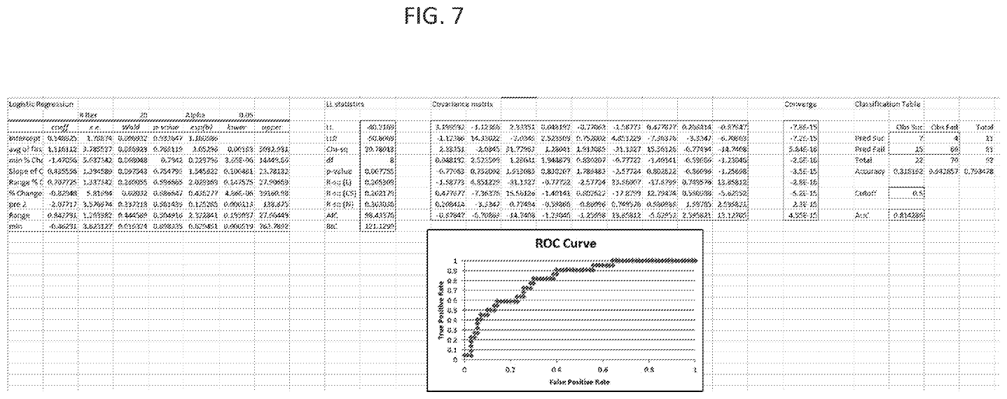

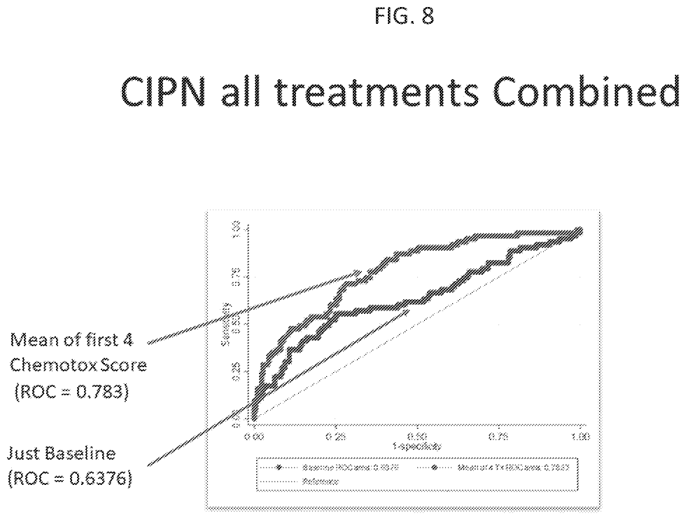

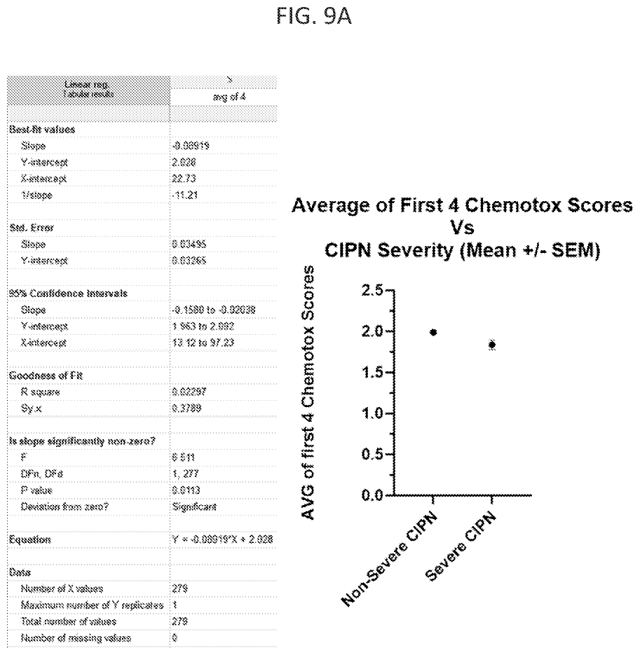

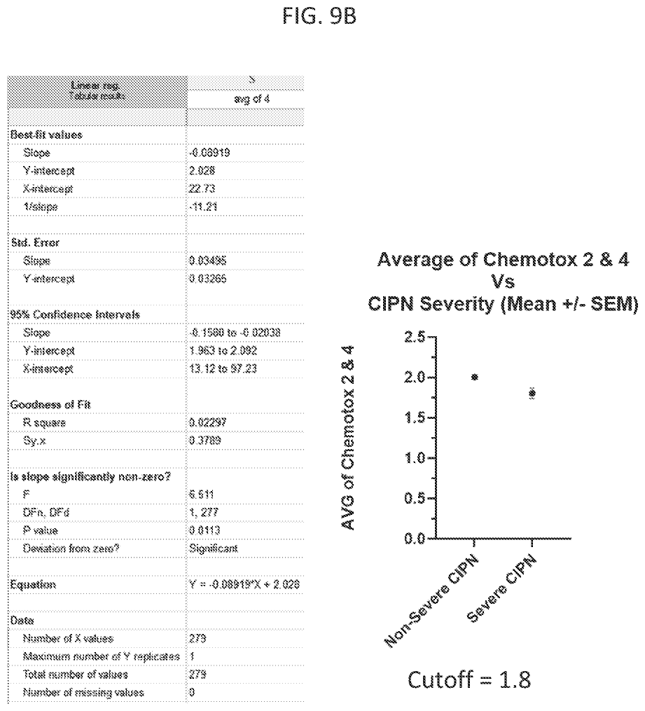

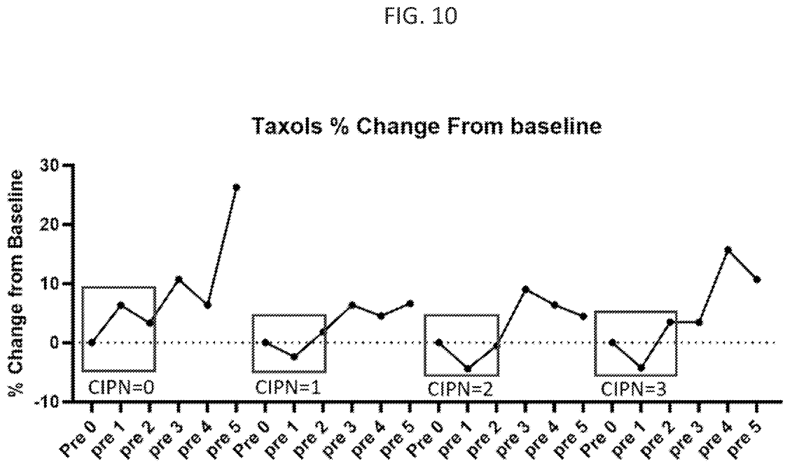

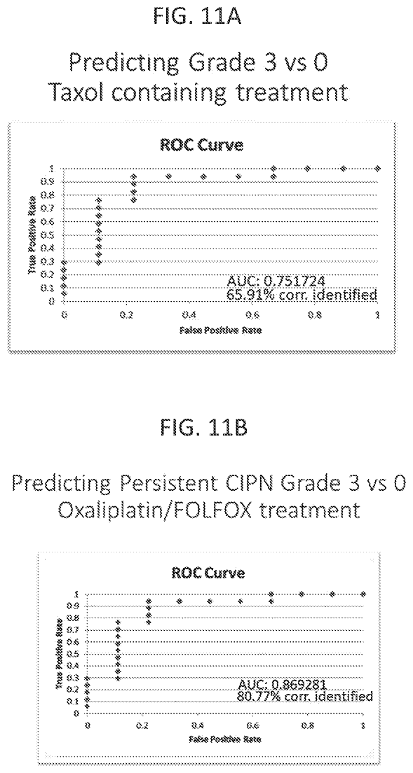

Results: We have enrolled 428 patients with an average age of 63.84 years (12.85 STDEV+13.00; Range; 25-92). Patients are predominantly of Caucasian heritage (82%) along with African American (16.59%) and Asian (0.43%), and Latino (0.48%) heritages. Females constitute 57.94% of the cohort. To date there are 306 patients with more than 24 months of follow-up and among these patients 19.28% reported Grade 3 CIPN and 24.51% reported Grade 2 symptoms, while 32.35% had no reported symptoms of CIPN. FIG. 2 shows that patient age is a significate risk factor for CIPN. Analysis of Chemotox scores based on gender and time of year showed that men generally have lower chemotox scores than women (i.e., lower levels of glutathione recycling) and levels can fluctuate different times of the year (FIG. 3). An initial analysis of a collection of four Chemotox scores for patients revealed that the combination of timepoints can be useful for determining a patient's susceptibility to CIPN (FIG. 4 and FIG. 8). Predictions of CIPN were obtained 5 to 12 weeks prior to onset of grade 2 or 3 CIPN symptoms. Of patients with persistent CIPN, defined as lasting more than 3 treatment cycles or follow-up visits, 80.77% were correctly identified using this method (AUC 0.869281) (FIG. 8). Using a baseline/pre-treatment measurement alone was not effective to predict severe CIPN as revealed by an AUC of 0.47 (FIG. 5). Alternatively, using a measurement obtained following a first treatment (and prior to a second treatment) yielded an AUC of 0.74 (FIG. 6). Next, it was evaluated whether severe CIPN could be predicted using a combination of Chemotox scores obtained from a patient a various timepoints and additional factors such as patient age and gender. Using an average of four measurements (1=pre-treatment/baseline, 2=following a first treatment/prior to a second treatment, 2=following a second treatment/prior to a third treatment, and 4=following a third treatment/prior to a fourth treatment) and additional criteria, the AUC for the assessment was improved to 0.81 (FIG. 7). Further analysis showed that average chemotox scores could be used to predicted severe CIPN in patients that underwent chemotherapy regimens. Using an average of the four Chemotox scores revealed a cutoff of 1.85 for predicting severe CIPN (FIG. 9A). Surprising, utilizing just an average of a Chemotox score obtained following a first treatment/prior to a second treatment and second score obtained and a Chemotox score obtained following a third treatment/prior to a fourth treatment was also highly predictive of sever CIPN with a cutoff of 1.8 (FIG. 9B). FIG. 11A and FIG. 11B show that an analysis using an average of Chemotox scores obtained at four timepoints was useful could predict CIPN in cohorts receiving taxol-containing treatment or a combination oxaliplatin/FOLFOX treatment. Including additional risk factors/variables can be utilized in combination with Chemotox scores to improve prediction of CIPN. Factors include patient age, race, gender, the specific treatment regimen, cancer type, comorbidities, previous cancer and treatment history, body-mass-index, genetic factors, and red-blood cell count.

### Example 2: Improved Assay for Measuring Glutathione Recycling Capacity

An existing tube-based assay (Oxphos™ Cell Survival Assay Kit; Rockland, Inc.) for measuring recycling capacity of the antioxidant glutathione has been used to determine level of oxidative stress and adverse effects induced by reactive oxygen species. We modified this previous microcentrifuge tube-based assay into a 96-well high-throughput assay and replaced some of the old chemistry to ensure an accurate final measurement using an automated plate reader, as is typically found in CLIA certified clinical laboratories.

A first modification to the assay included using a V-shaped 96-well microplate for incubation of blood samples with the bioactive probe hydroxy-ethyl-disulfide. After standard incubation time, the blood cells sediment to the bottom of the wells, allowing the supernatant to be transferred to a new plate. To remove interfering material that might have been released from damaged cells, a slurry of nanobeads is added. The nanobeads are made of silica (SiO₂) and maghemite (γ-Fe₂O₃), or similar, created according to the Stober process (Stöber, Werner; Fink, Arthur; Bohn, Ernst (January 1968). “Controlled growth of monodisperse silica spheres in the micron size range”. Journal of Colloid and Interface Science. 26 (1): 62-69. Bibcode:1968JCIS26-62-S. doi:10.1016/0021-9797(68)90272-5). Beads have surface ligands that bind molecules which can interfere with the final optical measurement. By placing the plate on a magnetic separator unit, beads with ligand bound substances sediment at the bottom of wells and clarified supernatants can then be transferred to an optical bottom plate for measurement in plate readers at 412 nm using dithiobisnitrobenzoic acid as in the previous methodology (Oxphos™ Cell Survival Assay Kit; Rockland, Inc.)

The improvements facilitate high volume testing in a clinical laboratory where automated high-throughput instruments are utilized and allow for a faster determination of glutathione recycling capacity in patients.

### Protocol for Multiwell Assay

### Non-Kit Materials:

Two V-bottom 96-well Plate
One Flat-bottom 96-well plate
Equilibration/Wash Buffer: 125 mM Tris-HCL, 150 mM NaCl, 1 mM EDTA, pH 7.4
Pierce Glutathione Magnetic Agarose Beads. Cat. No. 78601
Dexter LifeSep 96F magnetic Plate Separator
0.9% Sodium Chloride

### Preparation of Magnabeads

1. For each patient add 100 μL of bead slurry (25 uL settled beads) into a 1.5 mL microcentrifuge tube.
   Note: Use a larger tube for multiple patients and increase volume of beads and Equilibrium/Wash Buffer respectively.
2. Add 400 μL of Equilibration/Wash buffer to the beads and vortex for 10 seconds.
3. Place the tube into a magnetic stand to collect beads against the side of the tube. Remove and discard the supernatant
4. Add 500 μL of equilibration/wash buffer to the tube. Vortex the beads for 10 seconds and collect the beads with the magnetic stand. Remove and discard the supernatant.
5. Add 400 μL of equilibration/wash buffer to the tube and vortex for 10 seconds.
   Note: Do not allow the beads to dry. If necessary, store the beads in the Equilibration/Wash Buffer.

### Reagents

Reagent #1: (HEDS) 2-Hydroxyethyl disulfide (CAS 1892-29-1, MW 154.25 g/mol)
Reagent #2: (PBS) Phosphate-Buffered Saline, pH 7.4 2.0 g KCL, 2.40 g KH₂PO₄, 80.0 g NaCl and 14.4 Na₂IPO₄×7H₂O dissolve in distilled water, adjust pH and adjust volume to 1 L.
Reagent #3: (SS Acid) [100 mM 5-Sulfosalicylyc acid dihydrate, BioXtra ≥99%][Sigma 57422-100G]
   CAS 5965-83-3, CH₆O₆S·2H₂O, MW 254.21 g/mol
      To make 500 ml—Dissolve 12.71 grams into dist. H₂O. Once dissolved—adjust volume up to 500 ml with more water.
Reagent #4: (GSH buffer) [0.1M NaH2PO4·0.5 mM EDTA, pH7.5]
   [Sodium Phosphate Monobasic Anhydrous USP, Fisher S397-500] CAS 7558-80-7, MW 119.96 g/mol NaH₂PO₄;
   [Ethylenediamine Tetra-acetic Acid (EDTA), Disodium salt, dehydrate USP, Fisher S312-500]CAS 6381-92-6, MW 372.24, C₁₀H₁₄N₂Na₂H 2H₂O
   To make 1 L—Dissolve 11.996 grams of Monobasic Sodium Phosphate. Add 0.1862 grams of EDTA and continue stir the solution. The pH might need to be adjusted to allow all EDTA dissolve.
   Once completely dissolved—adjust pH to 7.5.
Reagent #5: (DTNB) [10 mM 5,5′-Dithiobis (2-nitro-benzoic acid)]
   [Sigma D8130-10G]
      CAS 69-78-3; MW 396.35; C₁₃H₈N₂O₈S₂
   To make 250 ml—Dissolve 0.9908 gram of DNTB in Reagent 4.

### Multi-Well Assay

### Step 1

Set up a V-bottom 96-well plate with saline and Reagent 6 as shown in Table 1, making duplicates for 5 & 10 uL blood samples for a total of 5 wells per patient. Gently mix blood into each well and then cover the plate with microplate sealing film before incubation on a rocker for 2 hours.

TABLE 1

| Blood | Saline | Reagent 6 |
|---|---|---|
| (uL) | (uL) | (uL) |
| 0 | 100 | 5 |
| 5 | 95 | 5 |
| 10 | 90 | 5 |

### Step 2

After 2-hour incubation, transfer 75 μL of the supernatant from each well from step 1 into a new V-bottom 96-well plate containing 75 μL of the washed magnetic bead solution in each well and allow incubation for 10 minutes.

### Step 3

After incubation, place the V-bottom 96-well plate from Step 2 onto the Dexter magnetic plate for 30-60 seconds. The magnetic beads will be pulled to the bottom of each well. Transfer 10 uL of the supernatant from each well into a flat bottom 96-well plate containing Reagents 4 & 5 as shown in Table 2 for a total of 5 wells per patient and 1 blank.

TABLE 2

| Sample | Reagent 4 | Reagent 5 | Supernatant |
|---|---|---|---|
| Blank | 180 uL | 20 uL | 0 uL |
| Blood (0, | 170 uL | 20 uL | 10 uL |
| 5 & 10 uL) | | | |

Place the lid on the flat-bottom 96-well plate and read the absorbance at 412 nm. Subtract the blank from the experimental samples

### Example 3: Chemotox High Throughput Assay

An improved assay was developed to allow for the efficient analysis of large number of patient samples.

### Materials Needed Per Sample

Three empty 96-well plate frames
Two V-Bottom 8-well strips per sample
One flat-Bottom 8-well strip per sample
Four 8-well Strip Cap's per sample
Reagent #1: Hydroxyethyl Disulfide (HEDS) (CAS 1892-29-1, MW 154.25 g/mol)
Reagent #2: Trichloroacetic Acid (TCA)
Reagent #3: Color Reagent, Ellman's Reagent (DTNB) [10 mM 5,5′-Dithiobis (2-nitro-benzoic acid)] [Sigma D8130-10G] CAS 69-78-3; MW 396.35; C13H8N2O8S2
Reagent #4 (Glutathione buffer)
Lyophilized L-Cysteine Hydrochloride Monohydrate (LCHM) [Alfa Aesar: CAS 7048-04-6

Listed below are layouts for ready-to-use 8-well strips containing specified reagents for the assay (each three strips contain wells A-H as depicted in FIG. 12). One of strip is needed per patient sample, and single plate frame can hold up to twelve strips in parallel (8×12) to perform the assay on twelve patient samples at once.

### 8-Well Strip #1: V-Bottom

| | |
|---|---|
| A | blank - 210 μl of Reagent #4 |
| B | positive control (low - 25 μM): lyophilized LCHM |
| C | positive control (high - 75 μM): lyophilized LCHM |
| D | negative control- 10 μL HEDS + 200 μL saline |
| E and F | 10 μL HEDS + 180 μL saline |
| G and H | 10 μL HEDS + 140 μL saline |

### 8-Well Strip #2: V-Bottom

| | |
|---|---|
| A | blank - 210 μl of Reagent #4 |
| B and C | positive controls - 200 μl of Reagent #4 |
| D | negative control- 200 μl of Reagent #2 |
| E and F | 200 μl of Reagent #2 |
| G and H | 200 μl of Reagent #2 |

### 8-Well Strip #3: Flat-Bottom

| | |
|---|---|
| A | blank - 185 μl of Reagent #4 + 25 μL Reagent #3 |
| B and C | positive controls - 185 μl of Reagent #4 + 25 μL Reagent #3 |
| D | negative control- 185 μl of Reagent #4 + 25 μL Reagent #3 |
| E and F | 185 μl of Reagent #4 + 25 μL Reagent #3 |
| G and H | 185 μl of Reagent #4 + 25 μL Reagent #3 |

### Performing the Assay:

1. Add 210 μL of Reagent #4 to wells B & C of Plate #1
2. Add 20 μL whole blood in duplicate to wells E & F (containing 180 μL saline) and add 60 μL of blood to wells G & H (containing 140 μL saline)
3. Seal the plate with cap strips and place on shaker at 700 RPM for 2 hours to incubate at room temperature
   Note: Optimal performance is obtained at 21° C. If ambient temperature differs from 21° C. use the following formula to obtain final Chemotox Score:

Chemotox_{FINL}=(Chemotox_{INITIAL}−(0.159*T_{ACTUAL}))+(0.159*21)
4. After 2-hour incubation, centrifuge plate #1 at 2,000 G for 6 minutes, making sure the centrifuge is adequately balanced beforehand.
5. Carefully transfer 50 μL of supernatant from each well into corresponding wells in plate #2 without disrupting the pellet. Seal the plate with a new strip cap.
6. Centrifuge plate #2 at 2,000 G for 6 minutes, making sure the centrifuge is adequately balanced.
7. Transfer 30 μL of supernatant from plate #2 into plate #3 and wash pipette tips. Color change from clear to yellow is immediate.
8. Read absorbance value at 412 nm within 30 minutes

A side-by-side analysis of patient samples comparing results from the commercially available tube-based method with results obtained the high-throughput plate-based format revealed that plate-based assay was at least as, if not more, accurate (FIG. 13). The addition of a positive control (LCHM) further enhances the reliability of the assay.

Each and every patent, patent application, and publication is incorporated herein by reference. U.S. Provisional Patent Application No. 63/275,271, filed Nov. 3, 2021, and U.S. Provisional Patent Application No. 63/275,279, filed Nov. 3, 2021, are incorporated herein by reference. While the invention has been described with reference to particular embodiments, it will be appreciated that modifications can be made without departing from the spirit of the invention. Such modifications are intended to fall within the scope of the appended claims.

What is claimed is:

1. An assay for measuring GSH recycling capacity in a collection of samples containing RBC, the assay comprising
(a) combining each of the samples with a solution containing HEDS to obtain a collection of first volumes, and incubating said collection of first volumes;
(b) centrifuging the samples to remove RBCs and debris from suspension;
(c) obtaining an aliquot of each of the said first volumes, substantially free of RBC and debris, and admixing each aliquot with trichloroacetic acid (TCA) to obtain a collection of second volumes;
(d) subjecting the collection of second volumes, or aliquots thereof, to centrifugation;
(e) obtaining a collection of supernatants from the second volumes, and combining each of the supernatants, or aliquots thereof, with a solution containing 5,5′-Disulfanediylbis(2-nitrobenzoic acid) (DNTB) to obtain a collection of third volumes; and
(f) subjecting the collection of third volumes, or aliquots thereof, to spectrophotometric analysis to obtain a series of absorbance readings,
wherein the absorbance readings obtained in (e) are indicative of GSH recycling capacity in the sample containing RBC.

2. The assay according to claim 1, wherein step (a) comprises obtaining a collection of first volumes in a series of 8-well strips of wells, optionally wherein each strip contains a different sample or dilutions of different samples.

3. The assay according to claim 1 or 2, wherein the series of 8-well strips include one or more wells containing a positive control comprising L-Cysteine hydrochloride monohydrate (LCHM), optionally in lyophilized form or in a solution at a concentration of about 25 μMol, about 50 μMol, and/or about 25 μMol to about 50 μMol.

4. The assay according to claim 2 or 3, wherein the 8-well strips include two or more different LHCM positive, each being a different amount or concentration of LCHM.

5. The assay according to any one of claims 2 to 4, wherein the 8-well strips include V-bottom wells.

6. The assay according to any one of claims 2 to 5, wherein the method of step (e) is performed with a collection of 8-well strips of wells, each strip including one or more of a blank, a sample, a positive control, and a negative control.

7. The assay according to any one of claims 2 to 6, step (e) is performed with using flat-bottom 8-well strips.

8. The assay according to any one of claims 2 to 7, wherein each of the 8 well-strips comprises:
(i) a blank;
(ii) a negative control volume;
(ii) one or two positive control volumes;
(iii) a lower blood sample volume, optionally in duplicate wells; and/or
(iv) a higher blood sample volume, optionally in duplicate wells.

9. The assay according to any one of claims 2 to 8, wherein the 8-well strips are placed into an frame capable of holding up to twelve 8-well strips during one or more steps including incubation, centrifugation, and spectrophotometric analysis.

10. An assay for measuring glutathione (GSH) recycling capacity in a sample containing red blood cells (RBC), the assay comprising
(a) combining the sample with a solution containing hydroxy-ethyl-disulfide (HEDS) to obtain a first volume, and incubating said first volume to allow a substantial fraction of RBC from the sample to sediment;
(b) obtaining an aliquot of the first volume, substantially free of RBC, and admixing said aliquot of the first volume with a solution containing magnetic nanobeads to obtain a second volume;
(c) subjecting the second volume, or an aliquot thereof, to a magnetic field;
(d) obtaining a supernatant from the second volume, and combining the supernatant, or aliquot thereof, with a solution containing 5,5′-Disulfanediylbis(2-nitrobenzoic acid) (DNTB) to obtain a third volume; and
(e) subjecting the third volume, or aliquot thereof, to spectrophotometric analysis to obtain an absorbance reading,
wherein the absorbance measured in (e) is indicative of GSH recycling capacity in the sample containing RBC.

11. An assay for measuring GSH recycling capacity in a collection of samples containing RBC, the assay comprising
(a) combining each of the samples with a solution containing HEDS to obtain a collection of first volumes, and incubating said collection of first volumes to allow a substantial fraction of RBCs from the sample to sediment;
(b) obtaining an aliquot of each of the said first volumes, substantially free of RBCs, and admixing each aliquot with a solution containing magnetic nanobeads to obtain a collection of second volumes;
(c) subjecting the collection of second volumes, or aliquots thereof, to a magnetic field;
(d) obtaining a collection of supernatants from the second volumes, and combining each of the supernatants, or aliquots thereof, with a solution containing 5,5′-Disulfanediylbis (2-nitrobenzoic acid) (DNTB) to obtain a collection of third volumes; and
(e) subjecting the collection of third volumes, or aliquot thereof, to spectrophotometric analysis to obtain a series of absorbance readings,
wherein the absorbances measured in (e) are indicative of GSH recycling capacity in the samples containing RBC.

12. The assay according to claim 10 or 11, wherein step (a) is performed in a v-bottom microplate.

13. The assay according to any one of claims 11 to 12, wherein step (e) is performed in a flat-bottom microplate.

14. The assay according to any one of claims 10 to 13, wherein step (e) is performed in an optical-bottom microplate.

15. The assay according to any one of claims 10 to 14, wherein the beads are made of silica and maghemite.

16. The assay according to any one of claims 10 to 15, wherein the assay is performed in a 96-well microplate.

17. The assay according to any one of claims 10 to 16, wherein the assay is performed in a 384-well microplate.

18. The assay according to any one of claims 10 to 17, wherein step (e) is performed using an automated plate reader.

19. The assay according to any one of claims 10 to 18, wherein the absorbance readings are performed at 412 nm.

20. The assay according to any one of claims 10 or 19, further comprising obtaining an absorbance reading of positive control comprising L-Cysteine hydrochloride monohydrate, optionally wherein the L-Cysteine hydrochloride monohydrate is present at a concentration of about 25 μMol, about 50 μMol, and/or about 25 μMol to about 50 μMol.

21. A kit for measuring glutathione (GSH) recycling capacity in a biological sample containing red blood cells, the kit comprising hydroxy-ethyl-disulfide (HEDS) and magnetic nanobeads,
optionally wherein the HEDS and/or the magnetic nanobeads are in a composition comprising a carrier.

22. The kit according to claim 21, wherein the magnetic nanobeads are glutathione magnetic agarose beads.

23. The kit according to claim 21 or 22, further comprising one or more of DTNB, a v-bottom plate, a flat-bottom plate, a magnetic separation unit, and L-Cysteine hydrochloride monohydrate (LCHM).

24. A kit for measuring glutathione (GSH) recycling capacity in a biological sample containing red blood cells, the kit comprising hydroxy-ethyl-disulfide (HEDS) and L-Cysteine hydrochloride monohydrate (LCHM),
optionally wherein the LCHM is in lyophilized form or in a solution at a concentration of about 25 μMol, about 50 μMol, and/or about 25 μMol to about 50 μMol.

25. The kit according to claim 24, further comprising one or more of DTNB, V-bottom 8-well strips, flat-bottom 8-well strips, a frame capable of holding up to twelve 8-well strips, trichloroacetic acid (TCA), and a glutathione buffer.
